(19) United States
(12) Patent Application Publication
(10) Pub. No.: US 2021/0339240 A1
(43) Pub. Date: Nov. 4, 2021

(54) ADD-ON DEVICE FOR ATTACHMENT ON A MICROPIPETTE

(71) Applicant: Panorama Laboratories B.V., Leiden (NL)

(72) Inventors: Jan Hendrik ZENDER, Leiden (NL); Maximillian Alan GREEN, Leiden (NL)

(73) Assignee: Panorama Laboratories B.V., Leiden (NL)

(21) Appl. No.: 17/280,152

(22) PCT Filed: Sep. 27, 2019
(86) PCT No.: PCT/NL2019/050652

§ 371 Date: Mar. 25, 2021

(30) Foreign Application Priority Data
Sep. 27, 2018 (NL) 1043014

(51) Int. Cl. B01L 3/02

(52) U.S. Cl. CPC B01L 3/0237; B01L 2300/023; B01L 2200/143; B01L 2300/0627; B01L 2300/027

(57) ABSTRACT

An add-on device for attachment to a micropipette for determining the rotational movement and longitudinal displacement of a micropipette plunger for digitization, communication and quantification of pipetting actions.

### TECHNICAL FIELD

The present invention relates to an add-on device for attachment on a micropipette.

### BACKGROUND ART

Many chemical and biochemical experiments require accurate volumetric handling of liquids in different concentrations in order to achieve reproducible and usable results. These experiments are often done in environments where extensive documentation is required, and thus, suction volume is expected to be documented accurately as well, since any deviation in suction volume can significantly impact reproducibility and accuracy. However, suction volume is, in most cases, only confirmed as an experimental step that is signed and not as an objectively measured data point.

There is currently a trend, that smart devices and networked systems provide more objective information in laboratory environments, providing information like pressure, temperature and humidity levels. However, data points that are of more direct influence on experimental data, like volumetric handling, remain largely undocumented. Since volumetric errors can quickly stack up to large deviations and it is not unusual for a single protocol to require many (e.g. 100) accurate volume mixing actions (often of small volumes under 100 microliters), objective data delivered by a smart device adds data integrity and experimental certainty.

There are options available to provide this objective suction volume data, mostly in the form of electronic or hybrid manual/electronic micropipettes. These devices are often costly to produce and vulnerable to impact and environmental changes due to the combination of—and interaction between—electronic and mechanical components. For instance, dropping a hybrid or electronic pipette can impact both the mechanics, as well as the electronic components that interact with the mechanics to produce readings. This extra vulnerability means that electronic pipettes, which are priced significantly higher than mechanical pipettes, are also much more likely to catastrophically fail and need replacement. Also, relative to mechanical devices, electronic devices are a lot more complicated to clean and service due to the increased number of parts and their sensitivity. This results in extra work and required knowledge for decontamination and servicing.

U.S. patent application US 2009/0055131 relates to a data acquisition apparatus which measures the rotational movement of a plunger in order to determine the volume setting of the pipette, and a switch for sensing whether the plunger has reached its most compressed position. A disadvantage of such a system is that there is no data acquired about the travel of the plunger over the longitudinal direction with respect to the plunger movement axis. In this way plunger movement speed is not monitored. The speed of the plunger may exceed acceptable speeds, thereby causing inaccuracies in pipetting. Another disadvantage of such a system is that the proposed system makes use of moving parts (switch) which are known to fail more often than nonmoving parts. The chance of failure of such a part increases over time. This would make data acquisition less reliable.

International patent application WO 2018/141898 is related to a micropipette that, among other things, outputs data regarding the angle of the pipette, pipetting actions and the turning of the plunger. A disadvantage of such a system is that the proposed device requires users to replace their existing functional pipettes with a new pipette for the sake of said data acquisition.

### OBJECTS OF THE INVENTION

One object of the present invention is to solve the problems as indicated above. This and other objects have been attained according to the present invention.

### SUMMARY OF THE INVENTION

The present technology provides for an add-on device for attachment on a micropipette. The add-on device comprises a sensor comprising means for sensing physical quantities, such as a magnetic field strength, ultrasound, light intensity or the compression of a helical spring. The sensor further comprises means for generating corresponding data. Furthermore, the add-on device further comprises a processing unit for processing said data and a communication unit, adjusted to provide communication means for communicating the data. Communication means include one of the following (but is not limited thereto): Bluetooth (including Bluetooth Low Energy), Wi-Fi, (wireless) serial communication, or any wired communication means such as ethernet, USB, I2C and FireWire. Furthermore, the add-on device may comprise a display unit, for displaying the data.

In one embodiment, the means for sensing physical quantities and generating corresponding data comprises a magnetometer for sensing at least one component of a magnetic field from a magnetic field source and for generating corresponding magnetic field data. Preferably, the add-on device further comprises an add-on magnetic source attached to the micropipette plunger, for providing the magnetic field. The add-on magnetic source may be, for instance, a permanent magnet or an electromagnet. Alternatively, the earth's magnetic field may be used as the magnetic source, in which case the magnetometer would sense local fluctuations in the earth's magnetic field caused by the movement of the plunger if the plunger is made from a material that interacts with the earth's magnetic field. The sensed magnetic field by the sensor may depend on a radial positioning of the magnetic source.

The means for sensing physical quantities and generating corresponding data may further comprise an accelerometer for sensing at least one component of an acceleration of the micropipette and for generating corresponding accelerometer data. Furthermore, the means for sensing physical quantities and generating corresponding data may further comprise a gyroscope, for sensing gyroscope data comprising at least one component of an angular velocity of the micropipette and for generating corresponding gyroscope data. The sensed gyroscope data and accelerometer data may depend on the user of the micropipette and the pipette tip management of the user of the micropipette. For this reason, the gyroscope data and/or the accelerometer data can be used to determine whether the micropipette has been used according to acceptable standards of said micropipette.

Preferably, the add-on device comprises a shielding material for shielding at least a part of the earth's magnetic field. The magnetic shielding material may for instance be made from a nickel-iron soft ferromagnetic alloy (such as trade name mu-metal®) or any other material with good magnetic field shielding properties.

In other embodiments, the sensor of the add-on device is further expanded with components for sensing mechanical, acoustical or optical data correlated to the plunger movement. The skilled person understands that the measured data from such sensor components can be correlated to the suction volume, in a similar manner as has been described for the embodiment wherein the sensor is a magnetometer. A suitable mechanical sensor component is a sensor component that employs a resilient member such as a helical spring. The extension or compression of the resilient member can be correlated to the suction volume. A suitable acoustic sensor component is an ultrasound sensor component, employing an ultrasound source. Accordingly, the ultrasound source is arranged in such a way that the acoustic sensor component measures the distance to a (bottom) part of the plunger that changes when a suction volume is being obtained or dispensed. A suitable optical sensor component is an interferometer. Accordingly, the interferometer is arranged in such a way that the interferometer measures the distance to a (bottom) part of the plunger that changes when a suction volume is being obtained or dispensed. Alternatively, instead of using an interferometer as optical sensor component, one could use at least one encoder to measure said distance to the (bottom) part of the plunger, or measure the amount of rotation the plunger has made.

The present technology further relates to a kit-of-parts, comprising an add-on device according to any of the previous described embodiments of an add-on device. The kit-of-parts further comprises a charging station for charging the add-on device.

The present technology is further related to a method for determining the volume setting of a micropipette, by using the add-on device according to any of the above described embodiments. Said add-on device is then attached to the micropipette. The method for determining the volume setting of a micropipette at least comprises the following steps: sensing at least one component of a magnetic field provided by either a magnetic source comprising a magnet mounted on the micropipette or the earth's magnetic field; generating corresponding magnetic field data; processing the magnetic field data such that a movement of a part of the micropipette can be extrapolated; communicating the magnetic field data and/or the processed magnetic field data.

### BRIEF DESCRIPTION OF THE ACCOMPANYING DRAWINGS

The present invention is described with respect to particular exemplary embodiments thereof and reference is accordingly made to the drawings in which:

FIG. 1 shows a schematic representation of an exemplary add-on device for attachment on a micropipette;

FIG. 2a shows a micropipette with a connected add-on device according to the present technology, wherein a plunger is in the extended or non-pressed arrangement;

FIG. 2b shows a micropipette with a connected add-on device according to the present technology, wherein a plunger is in the pressed arrangement;

FIG. 3a shows a micropipette with a connected add-on device and an attached magnetic source according to the present technology, wherein a plunger is in the extended or non-pressed arrangement;

FIG. 3b shows a micropipette with a connected add-on device and an attached magnetic source according to the present technology, wherein a plunger is in the pressed arrangement;

FIG. 4a shows an exemplary graph showing the relation of the magnetic field strength as a function of a distance to a magnetic source;

FIG. 4b shows an exemplary graph showing the relation of the magnetic field strength as a function of a suction volume;

FIG. 5a shows an example of a magnetic source of which the magnetic field is not radially uniform;

FIG. 5b shows an exemplary graph showing the relation of the magnetic field strength as a function of a distance to a magnetic source generating a magnetic field that is not radially uniform, wherein the magnetic source is moved with a screwing motion;

FIG. 6 shows a micropipette with add-on device according to the present technology implementing an optical sensor component;

FIG. 7 shows a micropipette with add-on device according to the present technology implementing a mechanical sensor component;

FIG. 8 shows a micropipette with add-on device according to the present technology implementing an acoustic sensor component;

FIG. 9a shows a micropipette with a connected add-on device and an attached magnetic source according to the present technology, wherein the magnetic source is radially asymmetric;

FIG. 9b shows an exemplary graph showing the relation of the magnetic field strength as a function of time wherein the magnetic field is not radially uniform and wherein the magnetic source is moved with a screwing motion at a constant rate.

The drawings show only those details essential to an understanding of the present invention.

### DETAILED DESCRIPTION

It will be appreciated that for simplicity and clarity of illustration, where considered appropriate, reference numerals may be repeated among the figures to indicate corresponding or analogous elements or steps. In addition, numerous specific details are set forth in order to provide a thorough understanding of the embodiments described herein. However, it will be understood by those of ordinary skill in the art that the embodiments described herein may be practiced without these specific details. Furthermore, this description is not to be considered as limiting the scope of the embodiments described herein in any way, but rather as merely describing the implementation of the various embodiments described herein.

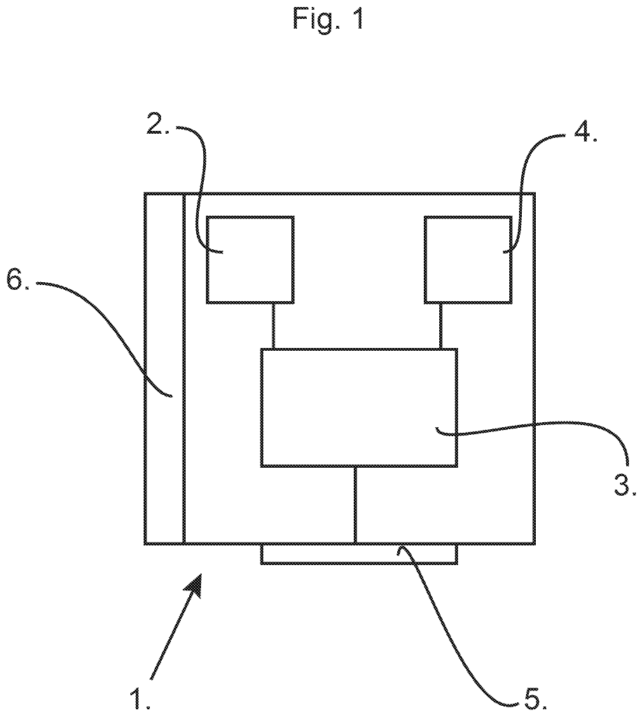

Reference is made to FIG. 1. FIG. 1 shows a schematic representation of an exemplary add-on device 1 for attachment on a micropipette. The add-on device 1 comprises a sensor 2 (which is further comprised of a number of sensor components), a processing unit 3, a communication unit 4 and a power source 5, preferably a (wirelessly) rechargeable battery, which is preferably rechargeable by means of a charging station. The add-on device 1 further comprises means for attachment 6 of the add-on device 1 to a micropipette.

In a preferred embodiment of add-on device 1, sensor 2 contains several sensing components of which at least one has the function of sensing the plunger movement relative to the micropipette body. In the preferred embodiment, other sensing components have the function of sensing movement and acceleration in three dimensional space of the micropipette itself. The skilled person understands that an advantage of this embodiment is that the additional data can be used for extrapolating micropipette tip management and user identification.

Furthermore, the skilled person understands that the additional data can be used to provide feedback to the user regarding use of the micropipette and whether the use is relevantly similar to the use as, for example, intended by the manufacturer of the micropipette.

Add-on device 1 comprises means to attach add-on device 1 to a micropipette. These means may include mechanical or chemical attachment 6, which may be permanent or temporary.

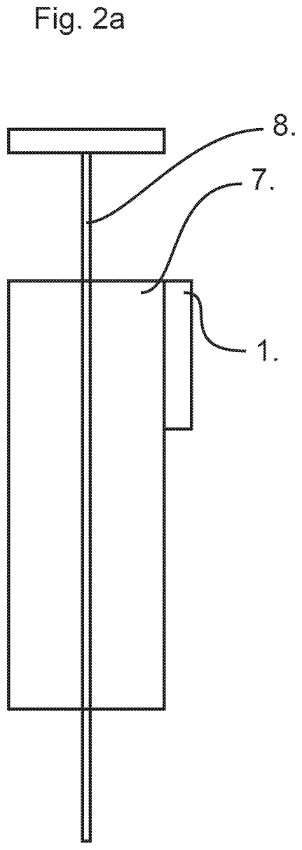

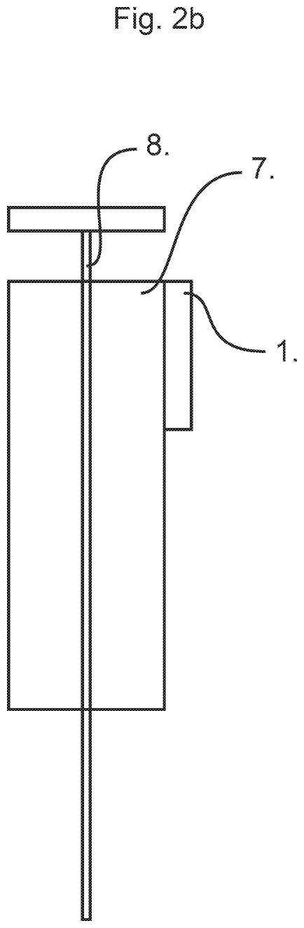

FIG. 2a shows a micropipette 7 on which the add-on device 1 of FIG. 1 is attached. Micropipette 7 may be any kind of pipette, for instance a displacement pipette such as air displacement pipette or a positive displacement pipette. The micropipette 7 comprises a plunger 8 which can move, for instance, in the longitudinal direction of the micropipette 7, for instance during use of the micropipette 7 for aspirating or dispensing a suction volume, for instance a liquid sample to be sampled.

The plunger 8 is made from one or more materials that influence the magnetic field. For instance, plunger 8 may be made from iron from which is known to be a paramagnetic material which locally influences the strength of a present magnetic field, such as the earth's magnetic field.

FIG. 2b shows the same micropipette 7 with add-on device 1, but now with the plunger 8 in a different position relative to add-on device 1. Due to the difference in positioning of plunger 8 relative to the add-on device 1, the sensor 2, comprising a magnetometer sensor component, senses a different magnetic field strength when compared to other positions of the plunger 8 relative to the add-on device 1. Sensor 2 generates data that represents the sensed strength of the local magnetic field. Sensor 2 is connected to the processing unit 3 and transfers the acquired data to the processing unit 3, which processes the transferred data, which may be analogue or digital data. Processing unit 3 processes the acquired data such that the data can be used to determine the suction volume. For instance, a predefined change in magnetic field may be correlated to a specific suction volume.

The processed data may be sent to an external processing unit such as a computer, a smartphone, or any other (smart) device, by means of the communication unit 4. Alternatively, the processing unit 3 directly transfers the data to said external processing unit.

An example of a suitable processing unit 3 is for instance a ATmega328 chip microcontroller. Examples of a suitable communication unit 4 include a chip that facilitates Bluetooth (including Bluetooth Low Energy), Wi-Fi, (wireless) serial communication, or any wired communication means such as ethernet, USB, I2C and FireWire.

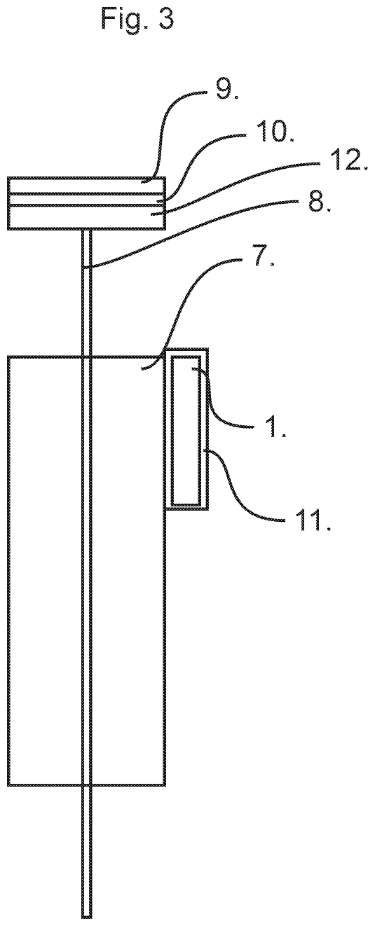

Reference is made to FIG. 3, which shows a preferred embodiment of the present technology. FIG. 3 shows a similar add-on device 1 for attachment on a micropipette, but the add-on device 1 now further comprises an add-on magnetic source 9. Add-on magnetic source 9 comprises means to attach add-on magnetic source 9 to a plunger 8 of a micropipette 7. These means may include mechanical or chemical attachment 10, which may be permanent or temporary. The skilled person in the art will understand what mechanical or chemical attachment 10 is suitable.

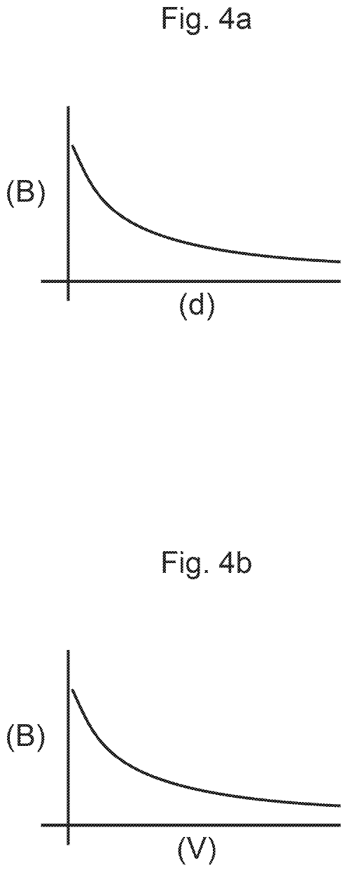

Processing unit 3 processes the acquired data such that the data can be used to determine the suction volume. For instance, a predefined change in magnetic field may be correlated to a specific suction volume. This is illustrated in FIG. 4a, which shows an exemplary graph of the acquired data by the magnetometer component of sensor 2, in this case local magnetic field (B), as a function of the relative distance (d) between a plunger head 12 of the plunger 8 and the add-on device 1. FIG. 4b shows the corresponding processed data, which has been processed by the processing unit 3 to correlate the distance (d) to a suction volume (V).

Magnetic source 9 may be, for instance, a permanent magnet or an electromagnet. In case of a permanent magnet, the magnet may be, for instance, shaped cylindrical, or may be shaped to correspond to a similar shape as the plunger head 12.

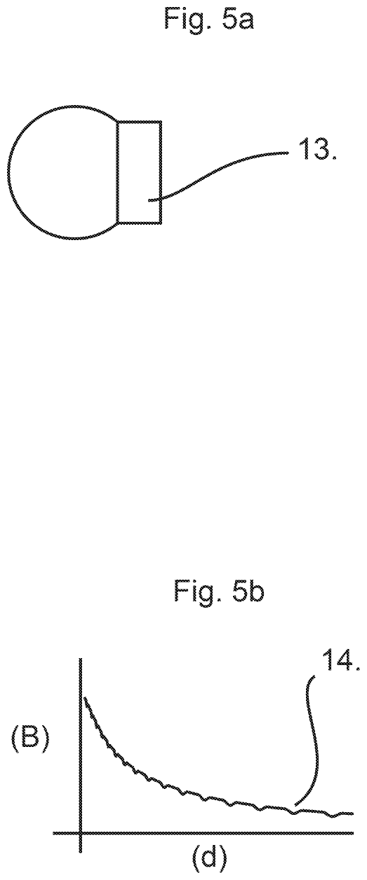

The magnetic source 9 may also be shaped such that the field as sensed by the magnetometer component of sensor 2 is not only dependent on the movement of the plunger 8, but also dependent on the radial positioning of the magnetic source 9. For instance, FIG. 5a shows a magnetic source 9 that comprises a permanent magnet with a cylindrical shape which has a protrusion 13 on one side of the permanent magnet. FIG. 5b shows an exemplary graph of the measured corresponding magnetic field (B) as measured by the magnetometer component of sensor 2, which is in this case placed off-axis relative to the magnetic source 9, as a function of distance (d) when the magnetic source 9 describes a screwing motion. This results in a regular fluctuation 14 in the magnetic field strength as sensed by the magnetometer component of sensor 2. An advantage of said configuration of the magnetic source 9 is that the amount of turning of the plunger 8 can be determined to further increase accuracy of the suction volume to be determined.

Add-on device 1 may further comprise a magnetic shielding material 11 that reduces and/or preferably eliminates the contribution of the earth's magnetic field to the measurement by the add-on device 1 of the magnetic field as provided by the magnetic source 9. The magnetic shielding material 11 may be made, for instance, from a nickel-iron soft ferromagnetic alloy (trade name mu-metal®) or any other material with good magnetic field shielding properties.

An advantage of using a magnetometer as a component of a sensor 2, as opposed to other proposed sensing components that have sensing the movement of plunger 8 as a goal, is the absence of moving parts within the add-on device. Thus, add-on device 1 using a magnetometer is less subject to wear and tear.

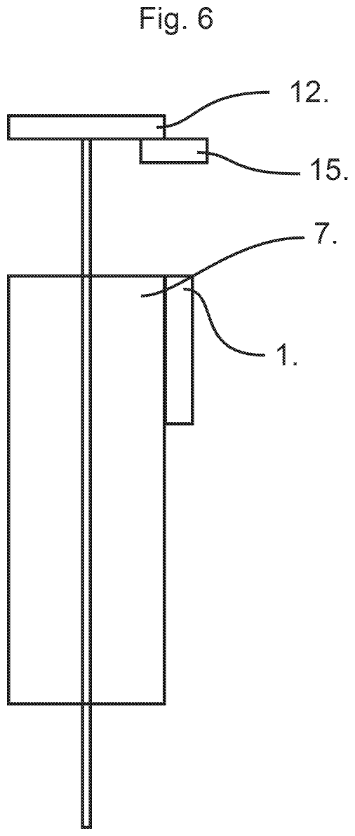

FIG. 6 shows a different embodiment of the present technology, wherein sensor 2 comprises an optical sensor component, such as an interferometer, and is arranged in such a way that the interferometer measures the distance to a part 15 of the plunger 8 that changes when a suction volume is being obtained or dispensed. If no such part 15 exists, such a part could be added to the plunger head 12 by chemical or mechanical attachment means. The skilled person understands that the measured distance data can be correlated to the suction volume, in a similar manner as has been described for the exemplary embodiment wherein the sensor 2 is a magnetometer.

Alternatively, instead of using an interferometer as optical component of sensor 2, one could use at least one encoder to measure said distance to part 15 of the plunger head 12, or measure the amount of rotation the plunger head 12 has made. The skilled person understands the possible arrangements of sensor 2 and part 15. The data from the encoder, or the measured amount of rotations can be correlated to the suction volume, in a similar manner as has been described for the exemplary embodiment wherein the sensor 2 comprises a magnetometer component.

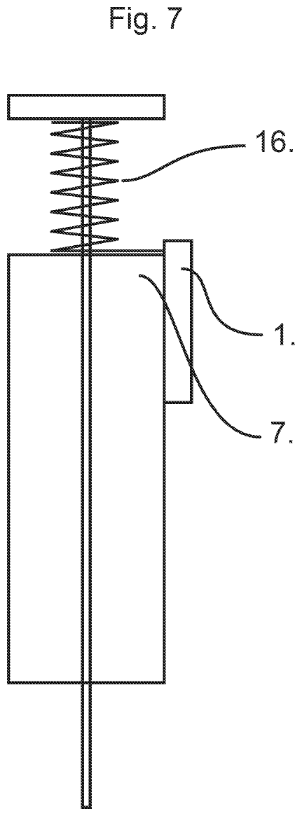

In yet another different embodiment of the present technology, sensor 2 comprises a mechanical sensor component. For example, FIG. 7 implements a mechanical sensor component comprising a resilient member 16, such as a helical spring. In this exemplary embodiment, sensor 2 measures the extension or compression of resilient member 16. The skilled person understands that the measured extension or compression values can be correlated to the suction volume, in a similar manner as has been described for the exemplary embodiment wherein the sensor 2 is a magnetometer.

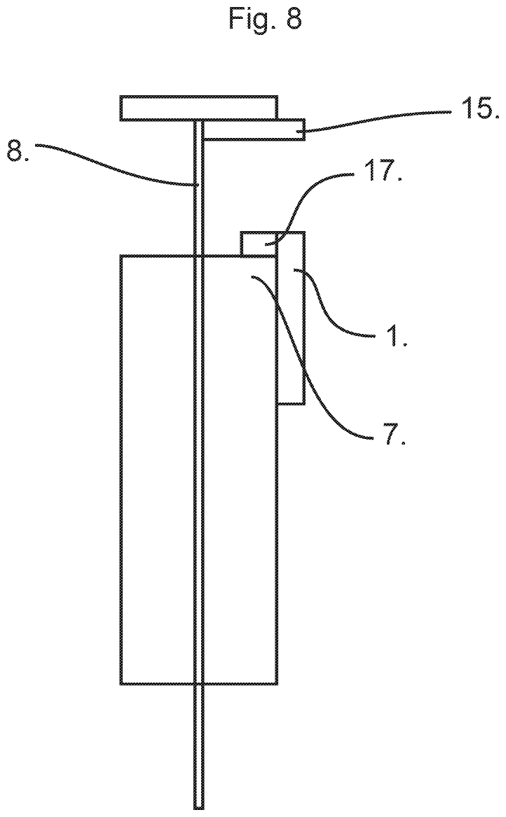

In yet another different embodiment of the present technology, sensor 2 comprises an acoustic sensor component. For instance, FIG. 8 shows an ultrasound distance measurement sensor comprising an ultrasound source 17, and is arranged in such a way that the acoustic sensor component of sensor 2 measures the distance to a part 15 of the plunger 8 that changes when a suction volume is being obtained or dispensed. If no such part 15 exists, such a part could be added to the plunger 8 by chemical or mechanical attachment means. The skilled person understands that the measured distance data can be correlated to the suction volume, in a similar manner as has been described for the exemplary embodiment wherein the sensor 2 comprises a magnetometer component.

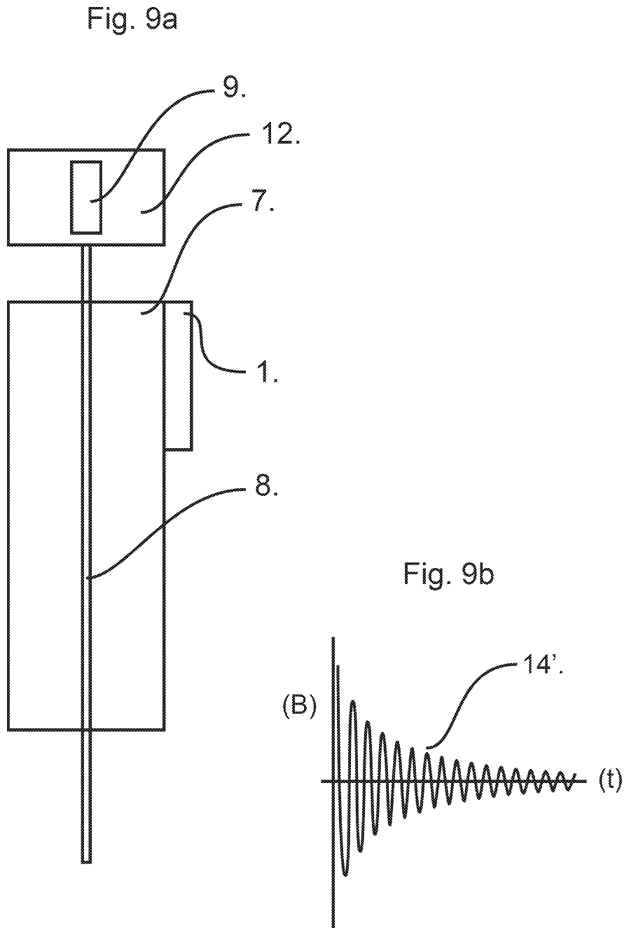

In yet another embodiment of the present technology, the suction volume of a micropipette 7 is determined by the number of turns of the plunger 8. FIG. 9a shows such an embodiment. The plunger 8 in this case comprises a screwed thread to determine the desired suction volume. The plunger head 12 may comprise a magnetic source 9.

Alternatively, plunger head 12 may be provided with an add-on magnetic source. The magnetic source 9 generates a magnetic field with primary components in the plane orthogonal to the longitudinal direction of plunger 8. In this embodiment, when plunger head 12 is turned, the magnetic field strength will alternate and change direction according to rotational positioning. FIG. 9b shows the magnetic field strength in the situation in which such a configuration of plunger 8 is turned at a constant rate over time. This allows determination of suction volume according to regular fluctuation 14′ after calibration procedures known by the person skilled in the art.

It is noted that the embodiments wherein sensor 2 comprises a magnetometer component is, compared to the other embodiments, less subject to interference with the relevant signal. Furthermore, the skilled person understands that the embodiments wherein sensor 2 comprises a magnetometer component are, compared to the other embodiments, easiest to install and service. Furthermore, the skilled person understands that a magnetometer component may require the least power to enable operation.

It is noted that an additional use of the presented embodiments, is that data and interpreted data from the add-on device can be presented to the user of the add-on device in a very short time-frame. This enables users of the add-on device to receive immediate feedback about the actions they have executed with the micropipette that the add-on device is added to. Research has indicated that a continuous stream of feedback regarding micropipette usage leads to a higher accuracy and precision in micropipette action quality.

The present invention has been described with regard to specific embodiments; however, it will be obvious to persons skilled in the art that a number of variants and modifications can be made without departing from the scope of the invention as described herein.

The clauses that follow define further embodiments forming part of the present disclosure.

Clauses

1. An add-on device for attachment on a micropipette, preferably a mechanical micropipette, comprising:
a sensor, comprising:
means for sensing physical quantities and generating corresponding data;
a processing unit for processing the data;
a communication unit, adjusted to provide communication means for communicating the data.

2. The add-on device according to clause 1, wherein the means for sensing physical quantities and generating corresponding data comprises a magnetometer for sensing at least one component of a magnetic field from a magnetic field source and for generating corresponding magnetic field data.

3. The add-on device according to clause 2, further comprising an add-on magnetic source, for providing the magnetic field.

4. The add-on device according to clause 3, wherein the add-on magnetic source comprises a permanent magnet.

5. The add-on device according to clause 4, wherein the sensed magnetic field by the sensor depends on a radial positioning of the magnetic source.

6. The add-on device according to clause 2, wherein the earth's magnetic field is used as the magnetic source.

7. The add-on device according to any of the preceding clauses, wherein the means for sensing physical quantities and generating corresponding data further comprises an accelerometer for sensing at least one component of an acceleration of the micropipette and for generating corresponding accelerometer data.

8. The add-on device according to any of the preceding clauses, wherein the means for sensing physical quantities and generating corresponding data further comprises a gyroscope, for sensing gyroscope data comprising at least one component of an angular velocity of the micropipette and for generating corresponding gyroscope data.

9. The add-on device according to any of the preceding clauses, further comprising a shielding material for shielding at least a part of the earth's magnetic field.

10. The add-on device according to any of the preceding clauses, further comprising a display unit for displaying the data.

11. The add-on device according to any of the preceding clauses, wherein the communication means comprises a chip that facilitates at least one of the following communication standards: Bluetooth (including Bluetooth Low Energy), Wi-Fi, (wireless) serial communication, or any wired communication means such as ethernet, USB, I2C and FireWire.

12. Method for determining an amount of suction volume from a micropipette, by using the add-on device according to any of the preceding clauses, wherein the add-on device is attached to the micropipette.

13. Method for determining an amount of suction volume from a micropipette, preferably a mechanical micropipette, comprising the steps of:
sensing at least one component of a magnetic field provided by either a magnetic source comprising a magnet mounted on the micropipette or the earth's magnetic field;
generating corresponding magnetic field data;
processing the magnetic field data such that a movement of a part of the micropipette can be extrapolated;
communicating the magnetic field data and/or the processed magnetic field data.

14. Kit-of-parts, comprising:
an add-on device according to any of the preceding clauses, wherein the add-on device further comprises a rechargeable battery;
a charging station for charging the rechargeable battery.

1. An add-on device for attachment to a micropipette, comprising:
a sensor comprising a magnetometer for sensing at least one component of a magnetic field of a magnetic source for generating corresponding magnetic field data for determining a rotational movement and longitudinal displacement of a micropipette plunger;
a processing unit for processing the magnetic field data;
a communication unit, adjusted to provide communication means for communicating the magnetic field data;
means for attachment of the add-on device to a micropipette;
an add-on magnetic source comprising a permanent magnetic, for providing the magnetic field;
wherein the sensed magnetic field by the sensor depends on a radial positioning of the magnetic source; and
wherein the add-on magnetic source is shaped such that the magnetic field as sensed by the sensor is not only dependent on the movement of the micropipette plunger, but is also dependent on the radial positioning of the magnetic source.

2.-5. (canceled)

6. The add-on device according to claim 1, wherein earth's magnetic field is used as the magnetic source.

7. The add-on device according to claim 1, wherein the sensor further comprises an accelerometer for sensing at least one component of an acceleration of the micropipette and for generating corresponding accelerometer data.

8. The add-on device according to claim 1, wherein the sensor further comprises a gyroscope, for sensing gyroscope data comprising at least one component of an angular velocity of the micropipette and for generating corresponding gyroscope data.

9. The add-on device according to claim 1, further comprising a shielding material for shielding at least a part of earth's magnetic field.

10. The add-on device according to claim 1, further comprising a display unit for displaying the magnetic field data.

11. The add-on device according to claim 1, wherein the communication unit comprises a chip that facilitates at least one of one or more wireless communication standards, and any wired communication means.

12. A method for determining a volume setting of a micropipette, by using the add-on device according to claim 1, wherein the add-on device is attached to the micropipette.

13. A method for monitoring a movement of a plunger of a micropipette, the method comprising the steps of:
determining a rotational movement and longitudinal displacement movement of the micropipette plunger by sensing at least one component of a magnetic field provided by a magnetic source comprising a magnet mounted on the micropipette or by sensing local fluctuations in earth's magnetic field;
generating corresponding magnetic field data;
processing the magnetic field data to generate processed magnetic field data such that at least one of the rotational movement and the longitudinal displacement movement of the micropipette plunger can be extrapolated; and
communicating at least one of the magnetic field data and the processed magnetic field data.

14. The add-on device according to claim 7, further comprising a display unit for displaying at least one of the magnetic field data and the accelerometer data.

15. The add-on device according to claim 8, further comprising a display unit for displaying at least one of the magnetic field data and the gyroscope data.

16. The add-on device according to claim 1, wherein the communication unit is configured to communicate using at least one of a wireless communication standard and a wired communication standard.

17. The add-on device according to claim 16, wherein the wireless communication standard comprises at least one of Bluetooth, Bluetooth Low Energy, Wi-Fi, and wireless serial communication.

18. The add-on device according to claim 16, wherein the wired communication standard comprises at least one of Ethernet, Universal Serial Bus (USB), I2C, and FireWire.
